(19) United States
(12) Patent Application Publication
(10) Pub. No.: US 2026/0198165 A1
(43) Pub. Date: Jul. 9, 2026

(54) FUNCTIONALIZED DIACETYLENE MONOMERS, CORRESPONDING POLYDIACETYLENES AND USES IN CAPACITIVE PHOTODETECTORS

(71) Applicant: B.G. NEGEV TECHNOLOGIES & APPLICATIONS LTD., AT BEN-GURION UNIVERSITY, Beer Sheva (IL)

(72) Inventors: Raz JELINEK, Modi'in-Maccabim-Re'ut (IL); Nitzan SHAULOFF, Be'er Sheva (IL)

(21) Appl. No.: 19/130,504

(22) PCT Filed: Nov. 15, 2023
(86) PCT No.: PCT/IL2023/051182

§ 371 Date: May 15, 2025

Related U.S. Application Data
(60) Provisional application No. 63/426,046, filed on Nov. 17, 2022.

(51) Int. Cl. H10K 30/35; C07C 233/44; C08F 38/00; C09D 149/00

(52) U.S. Cl. CPC H10K 30/35; C07C 233/44; C08F 38/00; C09D 149/00

(57) ABSTRACT

A bis (diacetylene) anthraquinone compound of Formula IA: wherein n1, n2, n3 and n4 are integers independently selected from 3 to 12 and each of L1 and L2 is a linkage, is provided. The corresponding polydiacetylene, electrodes coated with the polydiacetylene and C-dots, and capacitive organic photodetector based thereon, are also described.

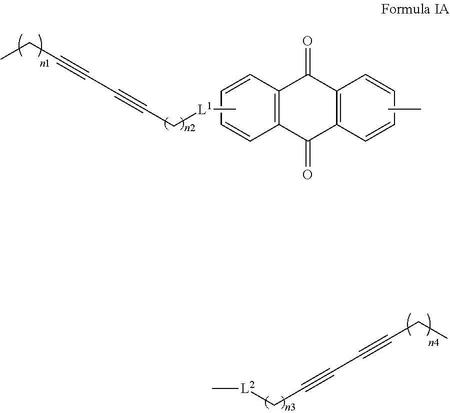

### BACKGROUND OF THE INVENTION

Multispectral photodetectors (MSPs) are widely used in optical communications, security systems, imaging, household electronics, molecular identification, and other applications. Traditional MSPs are based on semiconductor materials grown by epitaxial techniques which are generally not compatible with low-cost large-scale processing. Additionally, the performance and applicability of MSPs in desired spectral ranges mostly depend on the selection and physical tuning of the band-gap materials employed, thus presenting a challenging task. Moreover, the intrinsic properties of MSPs based on inorganic materials pose significant challenges in modern applications, as they exhibit limited biocompatibility, inadequate mechanical flexibility, and relatively high weight.

MSPs based on organic materials, also referred to as organic photodetectors (OPDs), may provide promising technological avenues. Recent studies reported on-par or better performance of OPDs compared to their inorganic counterparts, in terms of wavelength specificity, sensitivity, and size/weight. Organic MSPs employing photoactive polymers, small semiconductor organic compounds, cholesteric liquid crystals or nanocomposite carbon-based materials have been reported. While OPDs exhibit in many instances good wavelength tunability, cost-effective manufacturability, and simple fabrication processes, they have not yet gained sufficient commercial acceptance.

Capacitive photodetectors constitute an interesting class of OPDs. Capacitive photodetectors based on semiconductor-polymer composites and single-walled carbon nanotube (SWCNT) layers display efficient photon absorption and low power consumption (see S. Jun, S. bin Choi, C. J. Han, Y. T. Yu, C. R. Lee, B. K. Ju, J. W. Kim, ACS Appl Mater Interfaces 2019, 11, 4416 and S. Smirnov, I. V. Anoshkin, A. Generalov, D. V. Lioubtchenko, J. Oberhammer, RSC Adv 2019, 9, 14677). Conceptual and technical challenges for the applicability of capacitive photodetector technologies include slow response and recovery times, low sensitivity and complex fabrication processes that are incompatible with large-scale fabrication.

Carbon dots (C-dots), unique carbonaceous nanoparticles, have emerged as useful and versatile nanomaterials in opto-electronic applications due to their tunable optical and electronic properties. C-dots' photoluminescence can be modulated primarily through surface modification of the nanoparticles. Importantly, C-dots are inexpensive and easy to produce, they are biocompatible and non-toxic, and can be integrated within varied composite materials and matrixes. C-dots have been used as photosensitive components in photodetector designs. For example, Sarkar et al. [J Mater Chem C Mater 2019, 7, 13182] constructed broadband photodetectors comprising C-dots, reduced Graphene Oxide (rGO) and silver nanoparticles (Ag NPs) on p-doped silicon. The incorporation of C-dots within the rGO/Ag NP layers minimized the dark current and improved the PD responsivity. Q. Zhang et al. [ACS Nano 2015, 9, 1561] showed that the incorporation of C-dots within a UV photodetector facilitated the detection of light at very short wavelengths (down to 254 nm). C-dot-based photodetectors, however, have had limited applicability mostly due to cumbersome synthesis protocols, complex device fabrication processes, and often slow response/recovery times (see A. Subramanian et al., ACS Appl Electron Mater 2020, 2, 230; D. A. Nguyen et al., ACS Appl Mater Interfaces 2018, 10, 10322 and S. Miao et al., Front Energy Res 2021, 9).

Polydiacetylenes (PDAs) are π-conjugated organic polymers synthesized by crosslinking diacetylene (1,3-butadiyne-based monomers) under irradiation with ultraviolet (UV) light. PDAs are unique chromatic polymers, that exhibit visible colour transitions. As polymerized PDA generally appears blue due to the conjugated PDA network, it transforms to red when subjected to external stimuli. PDAs were used in varied optical sensing applications (e.g., X. Sun, T. Chen, S. Huang, L. Li, H. Peng, Chem Soc Rev 2010, 39, 4244). PDA systems have also been employed as a core component in the dielectric medium of capacitive sensors (V. K. Rao, N. Shauloff, X. M. Sui, H. Daniel Wagner, R. Jelinek, J Mater Chem C Mater 2020, 8, 6034).

Anthraquinones are derived from anthracenes and have two keto groups, mostly in positions 9 and 10, as shown below:

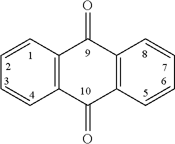 [SMILES: O=C1c2ccccc2C(=O)c2ccccc21]

Anthraquinone moieties have been employed in electrochemical applications, particularly via their participation in reversible redox reactions (W. Yin, A. Grimaud, I. Azcarate, C. Yang, J. M. Tarascon, Journal of Physical Chemistry C 2018, 122, 6546 and D. Bao, S. Ramu, A. Contreras, S. Upadhyayula, J. M. Vasquez, G. Beran, V. I. Vullev, Journal of Physical Chemistry B 2010, 114, 14467). It has been recently shown that polymerized monosubstituted anthraquinone-PDA manifests distinct aggregation-dependent chromatic properties, affected by the alignment of both the aminoanthraquinone headgroups and diacetylene sidechains (R. Bisht, V. Dhyani, R. Jelinek, Adv Opt Mater 2021, 9).

### The Invention

Experimental work reported below shows the synthesis of bis(diacetylene) monomers of Formula I:

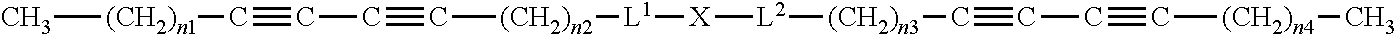

wherein X is a reducible aromatic group, each of L¹ and L² is independently a linkage and n₁, n₂, n₃ and n₄ are independently integers from 3 to 12, e.g., from 5 to 10. When the reducible unit X consists of the anthraquinone nucleus, the compound of Formula I has the structure depicted below:

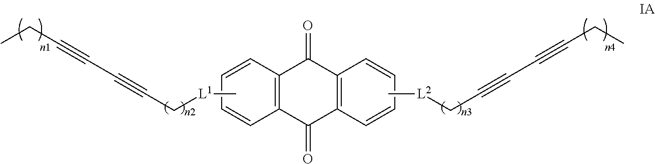

FIG. 1 shows the utility of one specific monomer of Formula IA. A thin film was produced by casting a solution of a monomer of Formula IA and C-dots on the surface of a commercially available interdigitated electrode (IDE) followed by ultraviolet irradiation to crosslink the adjacent diacetylene monomers, forming polydiacetylene network, with anthraquinone units in the polymer backbone chains and C-dots embedded across the polymer matrix. Scanning electron microscopy (SEM) image of the coated electrode shows a sheet-like surface morphology of the aligned polydiacetylene network. The coated electrode was shown to produce distinct light-induced capacitive signals that are sensitive to wavelength and circular polarization of the illuminating light beam (CPL detection). That is, the device constructed and shown in FIG. 1 is useful as a capacitive multispectral photodetector featuring good photo-responsiveness, i.e., exhibiting a significant change in capacitance in response to light illumination, with high sensitivity and wavelength selectivity over a broad spectral range (350 nm-650 nm). Experimental results reported below further indicate that the wavelength selectivity of the capacitive photodetector depends on the light absorbance profile of the C-dots employed, underscoring an intrinsic tunability of the photodetector system by the C-dots. Furthermore, the capacitive photodetector can distinguish circularly polarized light through enantioselective polymerization of the monomer of Formulas I/IA.

The bis(diacetylene) monomer of Formula IA shown above (especially a symmetric monomer, i.e., featuring the same diacetylene chains such that n₁=n₄, n₂=n₃) forms one aspect of the invention. The bis(diacetylene) monomer of Formula IA is prepared by a reaction between a diacetylene compound of Formula 2 and 9,10-anthraquinone of Formula 3:

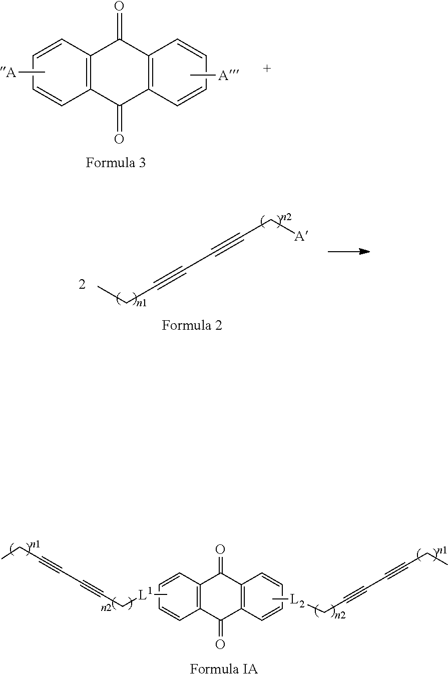

wherein n₁, n₂, L₁ and L₂ are as defined above, and A′, A″ and A″′ denote functional groups which can participate in a linkage formation reaction. That is, A′ participates in a linkage formation reaction with A″ and A″′ to create the linkages denoted L¹ and L², respectively, which may be the same or different. For example, each of the linkages L¹ and L² is an amide bond.

The A″ and A″′ groups are preferably attached at positions 1 and 5 of the 9,10-anthraquinone nucleus, and both are preferably amino (—NH₂) groups. A reaction between 1,5-diaminoanthraquinone and a diacetylene derivative of Formula 2 in which A′ is C(O)Cl (i.e., the acyl halide derivative of Formula 2) affords the preferred monomer of Formula IA with the structure depicted below (labeled IA-1):

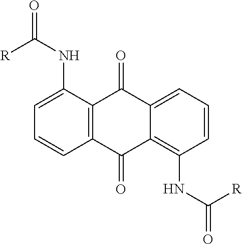

in which each R is CH₃—(CH₂)ₙ₁—C≡C—C≡C—(CH₂)ₙ₂—. Preferably,

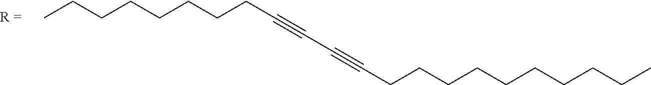 [SMILES: CCCCCCCCC#CC#CCCCCCCCCCC]

The most preferred compound of Formula IA-1, with n₁=9 and n₂=8, is shown below:

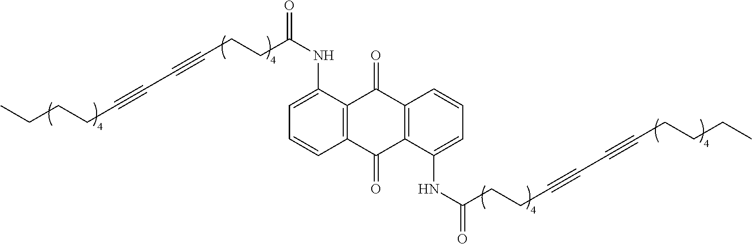

This monomer is labeled herein Bis-ADA.

Starting with the diacetylene monomers of Formula 2

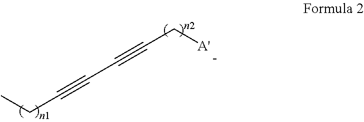

they are commercially available in the form of their carboxylic acid derivatives, i.e., A′=—COOH, e.g., 10,12-tricosadiynoic acid (TRCDA), 10,12-pentacosadiynoic acid, 10,12-octadecadiynoic acid, 5,7-docosadiynoic acid, 5,7-pentacosadiynoic acid and 5,7-tetracosadiynoic acid. The carboxylic acids are readily transformed into the corresponding acyl halide (—COCl). Acyl halides are more reactive reagents than the parent carboxylic acids and are more favorable for use in the invention. Acyl chloride of carboxylic acid is normally prepared by dissolving the carboxylic acid in an organic solvent, such as dichloromethane, using reagents such as oxalyl chloride (Cl—C(O)—C(O)—Cl), thionyl chloride (SOCl₂) or phosphorus trichloride (PCl₃). The reaction is advanced by the addition of a small, catalytically effective amount of dimethylformamide. The acyl chloride derivative of Formula 2 can be recovered after the removal of the solvents and excess reagent, for use in the preparation of the compound of Formula IA.

For example, the reaction of 10,12-tricosadiynoic acid (TRCDA) with oxalyl chloride in dichloromethane affords the corresponding acyl chloride of formula 2:

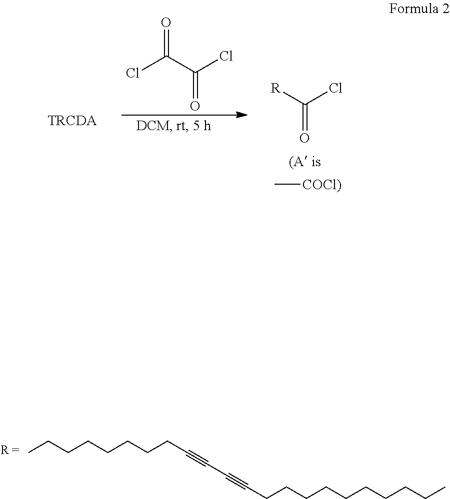

Turning now to the 9,10-anthraquinone represented by Formula 3:

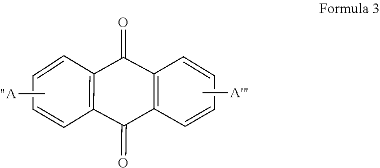

The α,α-disubstituted derivatives can be used in the invention, i.e., A″ and A″′ are attached at positions 1,5 or 1,8 of the anthraquinone nucleus. Preferred is α,α-diaminoanthraquinone, (each of A″ and A″′ is —NH₂), such as 1,5-diaminoanthraquinone, because the amino groups readily react with the acyl halide of Formula 2 to form an amide linkage (each of L¹ and L² is —NHC(O)—). 1,5-dihydroxyanthraquinone (each of A″ and A″′ is —OH) is yet another example of a starting material of Formula 3 that can be used, as the alcohol of Formula 3 reacts with the acyl halide of Formula 2 to form an ester linkage (each of L¹ and L² is —C(O)—O—). Mixed α,α-disubstituted derivatives (A″≠A″′) can also be used, e.g., 1-amino-5-hydroxy-anthraquinone. Suitable starting materials of Formula 3 are commercially available or can be prepared by methods known in the art, e.g., U.S. Pat. No. 3,933,868 showing the synthesis of 1,5-diaminoanthraquinone from the corresponding 1,5-dinitroanthraquinone and U.S. Pat. No. 3,773,800 showing the production of 1,5-dihydroxyanthraquinone.

However, the activation of the commercially available acid forms of the diacetylenes of Formula 2 (A′=COOH) to the acyl chloride is not essential and the acids may be reacted directly with α,α-diaminoanthraquinone to give the bis(diacetylene) monomers of Formula I/IA, under appropriate catalytic conditions (see a recently published review on the catalysis of such reactions: Taussat, A.; de Figueiredo, R. M.; Campagne, J.-M. Direct Catalytic Amidations from Carboxylic Acid and Ester Derivatives: A Review. Catalysts 2023, 13, 366. https://doi.org/10.3390/cata113020366 Academic Editors).

Thus, another aspect of the invention is a process comprising a reaction between a diacetylene monomer of Formula 2 and anthraquinone of Formula 3, as depicted below:

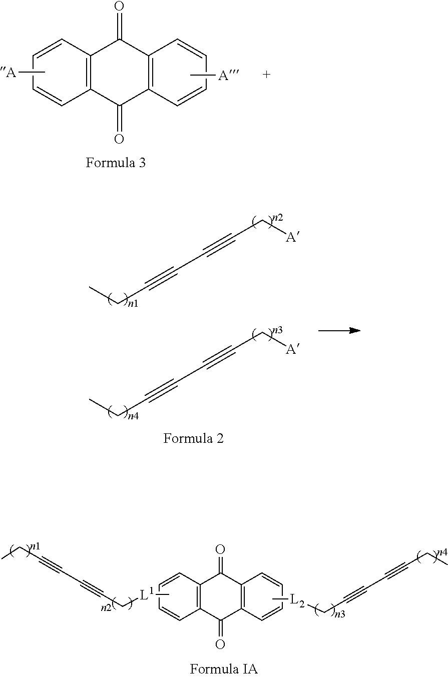

Specifically, for the preparation of symmetric monomers of Formula IA (n₁=n₄, n₂=n₃):

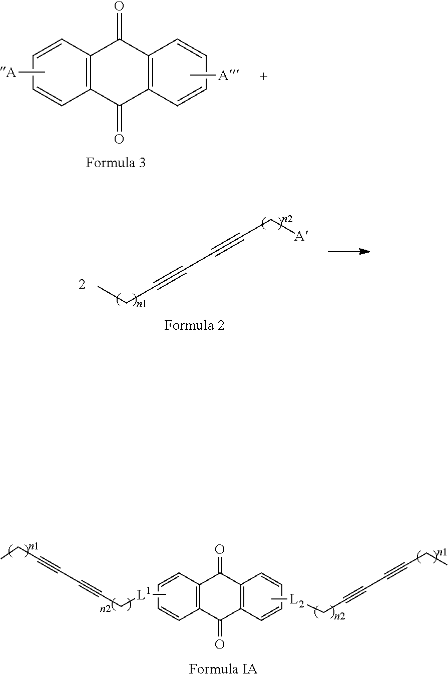

e.g., a base-catalyzed amide formation reaction wherein A″ is —NH₂, A″′ is —NH₂, A′ is —C(O) Cl and each of L¹ and L² is amide bond —NH—C(O)—. The acyl halide of Formula 2 is usually employed in excess relative to the amount dictated by stoichiometry (i.e., at least 10% molar excess above the 2:1 stoichiometric ratio, e.g., >2.2:1 ratio).

For example, the synthesis of the monomer of Formula IA from the acyl chloride of Formula 2 and 1,5-diaminoanthraquinone of Formula 3 can take place in an organic solvent (dichloromethane is a good choice) in the presence of a base; pyridine is often used as a co-solvent and a base removing protons formed in the reaction and activating the carbonyl group (Formula 2, A′ is —C(O)Cl) towards the attack by the amino groups (Formula 3, A″ and A″′ are both —NH₂).

For example, one specific process of the invention comprises gradual addition of a solution of acyl chloride of Formula 2 in dichloromethane to a reaction vessel that was previously charged with dichloromethane, pyridine and α,α-diaminoanthraquinone (e.g., 1,5-diaminoanthraquinone), allowing the reaction to reach completion (usually with stirring at room temperature) to form the corresponding bis(diacetylene) of Formula IA and working up the reaction mixture to recover the bis(diacetylene) of Formula IA:

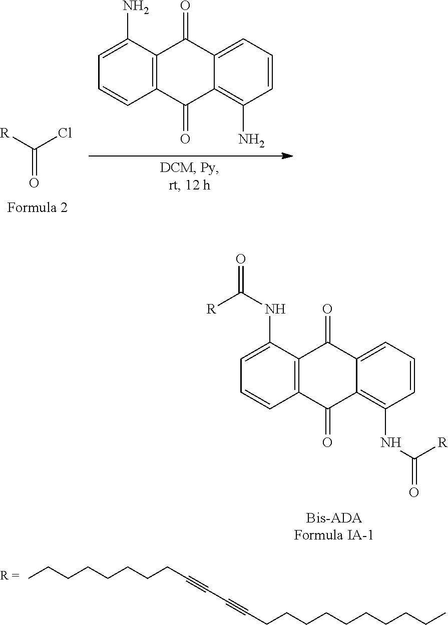

The working-up of the reaction mixture includes concentration of the solution by removal of the solvent, e.g., evaporation of dichloromethane under vacuum, addition of an antisolvent (e.g., water), separation of the product by filtration and subsequent washing (with water), purification by recrystallization from ethanol following which the collected precipitate is dissolved in an organic solvent such as halogenated alkane (e.g., chloroform), and the solution is concentrated under vacuum. The crude collected product can be purified by column chromatography over silica gel using e.g., ethyl acetate/toluene. The bis(diacetylene) of Formula IA, for example, Bis-ADA, is recovered in the form of a powder.

Polydiacetylene, comprising a repeat unit corresponding to the bis(diacetylene) monomers of Formula I/IA described above, form another aspect of the invention.

Carbon dots needed for the fabrication of the capacitive photodetector of the invention can be prepared by the solvothermal method described by L. Wang, W. Li, L. Yin, Y. Liu, H. Guo, J. Lai, Y. Han, G. Li, M. Li, J. Zhang, R. Vajtai, P. M. Ajayan, M. Wu, Full-color fluorescent carbon quantum dots. Sci. Adv. 6, eabb6772 (2020). A mixture of o-phenylenediamine

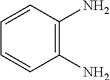

and a mild acid in ethanol solvent is maintained under heating (>150° C., e.g., at 180° C.) for several hours in a pressure reactor (e.g., a stainless-steel autoclave). Mild acids such as 4-aminobenzenesulfonic acid, folic acid, boric acid, acetic acid, terephthalic acid, and tartaric acid can be used in the solvothermal reaction to produce carbon dots emitting full-color fluorescence spanning the entire visible range under UV light irradiation. For example, o-phenylenediamine and an acid (e.g., folic acid (FA), boric acid (BA) or terephthalic (TPA) are added at roughly equal weights to ethanol solution which is introduced into an autoclave held at high temperature (e.g., 180° C.) for several hours (e.g., 12 hours). At the end of the solvothermal synthesis, the reaction mixture is cooled to room temperature, and the C-dots are separated by filtration from the liquid phase. Following purification (e.g., by dialysis in ethanol) and drying, a powder is collected which is suitable for use in the invention.

The synthesis of C-dots through hydrothermal treatment of ortho-phenylenediamine in 1) ethanol/FA, 2) ethanol/BA, and 3) ethanol/TPA mixtures and the normalized fluorescence spectra of the three C-dots produced (blue C-dots, green C-dots, and red C-dots, respectively) are shown in the experimental section below. The solvothermal syntheses yielded C-dots exhibiting distinct colors (i.e., distinct fluorescence emissions; photographs showing the C-dot colors upon illumination at 365 nm are presented in FIG. 2i). The normalized fluorescence excitation spectra in FIG. 2ii underscore the specific wavelengths, in a broad spectral range, absorbed by the C-dots (maximal fluorescence excitations at 400 nm, 440 nm, and 580 nm for blue C-dots, green C-dots, and red C-dots, respectively).

To fabricate a capacitive photodetector, a mixture consisting of reducible bis(diacetylene) monomer (e.g., Formulas I, IA or IA-1) and C-dots is applied onto the surface of an electrode, followed by UV light irradiation to bring about polymerization and form the polydiacetylene-coated electrode.

Experimental work conducted in support of this invention highlights the key role of a reducible unit incorporated into the backbone chains of the polydiacetylene film, in conjunction with the carbon dots embedded across the film. Results shown below suggest that light illumination of the capacitive photodetector results in photons being absorbed by the C-dots, with excitation of electrons between the HOMO and LUMO levels of the C-dots. These ejected electrons are accepted by the reducible groups which form part of the repeat unit of the polydiacetylene network. The reducible groups constitute efficient electron acceptors, undergoing redox transformations. That is, without wishing to be bound by theory, it is assumed that the reduction process occurring due to photoinduced electron transfer from the C-dots accounts for the significant, rapid capacitance changes shown by the system.

Accordingly, another aspect of the invention is a process for preparing a capacitive organic photodetector, comprising the step of polymerizing on an electrode surface a reducible bis(diacetylene) monomer in the presence of carbon dots, thereby forming a polydiacetylene film coating onto the electrode, with the carbon dots being embedded in the film.

A reducible bis(diacetylene) monomer of Formula I can be used:

CH₃—(CH₂)ₙ₁—C≡C—C≡C—(CH₂)ₙ₂-L¹-X-L²-(CH₂)ₙ₃—C≡C—C≡C—(CH₂)ₙ₄—CH₃ (I)

wherein X is a reducible aromatic group, each of L¹ and L² is independently a linkage and n₁, n₂, n₃ and n₄ are independently integers from 3 to 12. X is preferably a reducible polycyclic aromatic group consisting of two or more rings fused together, with at least one ring bearing one or more oxo (═O) groups attached to ring carbon(s). For example, the reducible group X is 9,10-anthraquinone, e.g., a monomer of Formula IA is especially suitable for use in the fabrication of the capacitive photodetector:

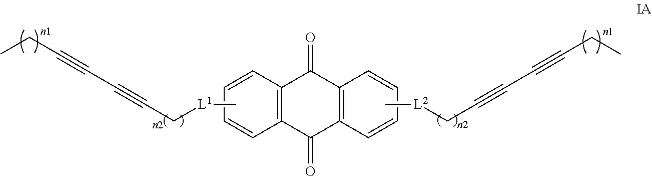

wherein each of L¹ and L² is an amide bond, for example:

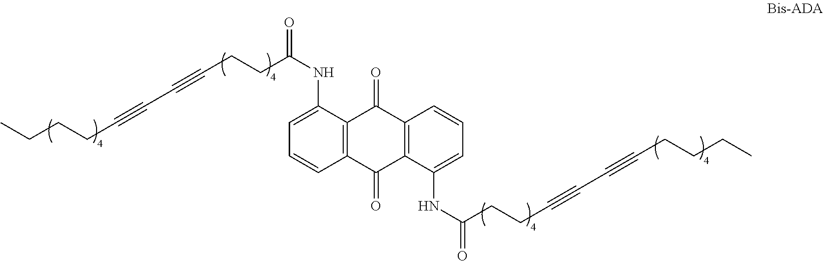

One useful configuration of the capacitive organic photodetector is based on interdigitated electrodes (IDEs). IDEs are usually patterned on an insulating substrate (by techniques such as photolithography and metal deposition) to create a comb-like configuration, i.e., two opposing interlocking comb-structured metal electrodes that are patterned onto the insulating substrate. The fingers of the electrodes are equally spaced apart, e.g., the gap between each two adjacent fingers is from 1 to 10 μm (usually there is a total of 20 to 100 fingers deployed on the substrate, e.g., glass substrate). But a capacitor with a conventional design consisting simply of two opposing electrodes can also be used. In both designs, the electrodes are made of metals such as gold, nickel, silver, iridium, platinum and from graphite.

Techniques such as solvent casting (e.g., drop-casting on a laboratory scale or doctor blade casting on a large scale), spray-coating and spin-coating can be used to create polymerizable films consisting of the bis(diacetylene) monomer of Formula I, specifically the bis(diacetylene) anthraquinone of Formula IA, and carbon dots evenly dispersed across the film.

For example, the diacetylene monomer and the carbon dots are combined in an organic solvent, and the resultant mixture is applied, e.g., by casting, onto the electrode surface, followed by polymerization by irradiation with non-polarized UV light to create the polydiacetylene film, or, simultaneously with the irradiation by non-polarized UV light, an irradiation with polarized light is carried out to create chiral polydiacetylene film (the difference between the two modes of UV irradiation depend on the contemplated utility of the capacitive photodetector, as explain below).

For example, toluene, owing to its ability to solubilize the bis(diacetylene) anthraquinone of Formula IA and its high volatility, is well suited for use in the generation of thin films by solvent casting. A solution of 1-20 (mg/mL) of the bis(diacetylene) anthraquinone of Formula IA is prepared and then the C-dots are added to the solution, usually at a weight ratio in the range from 1:1 to 1:5, from 1:2 to 1:3, followed by solution casting on the electrode. The film is allowed to dry, i.e., by evaporation of the solvent at room temperature, to create a 1-100 μm thick film on the electrode surface. Other organic solvents that may be used instead of alkylated benzene (e.g., toluene) include halogenated hydrocarbons such as dichloromethane and chloroform.

The resultant coated electrode is irradiated by UV light (less than 310 nm, e.g., 254 nm) to produce the polydiacetylene film.

The embedded C-dots do not disrupt the assembly and polymerization of the diacetylene monomers. Incorporation into the film of carbon dots that exhibit excitation fluorescence peak at a wavelength in the range of 300 nm<λ₁<420 nm (e.g., 380 nm<λ₁<420 nm), 420 nm<λ₂<500 nm or 500 nm<λ₃ produces a capacitive organic photodetector showing wavelength-dependent increase in capacitance in response to light illumination. That is, the invention provides a capacitive photodetector exhibiting a capacitance (C) that is sensitive to illumination with light of a specific wavelength (λᵢₗₗᵤₘᵢₙₐₜᵢₒₙ), as shown by ΔC/C (%) versus wavelength (λᵢₗₗᵤₘᵢₙₐₜᵢₒₙ) plots in the experimental section below. The wavelength dependence is directly linked to the absorbance wavelengths of the C-dots embedded in the polymerized bis(diacetylene) anthraquinone, as tabulated below:

TABLE 1

| | | significant |
|---|---|---|
| | | capacitance |
| | | response |
| | excitation | (% ΔC/C₀) |
| coating applied | fluorescence | is obtained |
| on the electrode | peak of the | when λ |
| surface | C-dots | illumination is |
| Polymerized | 300 nm < λ₁ < | 380 nm < λ |
| bis (diacetylene) | 420 nm | illumination < |
| anthraquinone/ | (380 nm < λ₁ < | 420 nm |
| blue C-dot | 420 nm) | |
| Polymerized | 420 nm < λ₂ < | 420 nm < λ |
| bis (diacetylene) | 500 nm | illumination < |
| anthraquinone/ | | 500 |
| green C-dot | | |
| Polymerized | 500 nm < λ₃ | λ illumination > |
| bis (diacetylene) | | 500 nm |
| anthraquinone/ | | |
| red C-dot | | |

Thus, another aspect of the invention is the process of preparing a capacitive organic photodetector showing a wavelength-dependent increase in capacitance in response to light illumination, comprising incorporating into the film carbon dots that exhibit excitation fluorescence peak at a wavelength in the range of 300 nm<λ₁<420 nm (380 nm<λ₁<420 nm), 420 nm<λ₂<500 nm or 500 nm<λ₃.

The invention further provides a capacitive organic photodetector comprising a polydiacetylene film-coated electrode with reducible sites along the polymer backbone and carbon dots embedded in the film, e.g., the backbone of polydiacetylene contains a reducible polycyclic aromatic group in the repeat unit. For example, the reducible polycyclic aromatic group is oxo-substituted, e.g., a quinone derivative, such as 9-10 anthraquinone. The backbone of the polydiacetylene preferably contains an amide bond. Specifically, the capacitive organic photodetector comprises a polydiacetylene film-coated electrode wherein the repeat unit of the polymer corresponds to:

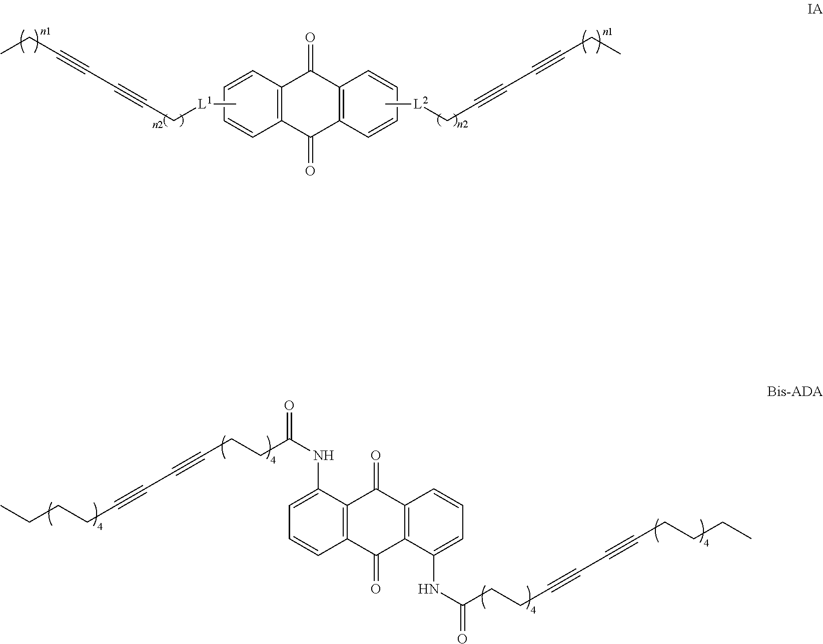

The capacitive organic photodetector of the invention shows wavelength-dependent capacitance change in response to light illumination. The invention specifically provides:

A capacitive organic photodetector wherein carbon dots embedded in the film exhibit excitation fluorescence peak at a wavelength in the range of 300 nm<λ₁<420 nm (e.g., 380 nm<λ₁<420 nm), with the photodetector being selectively responsive to light illumination at a wavelength in said range;

A capacitive organic photodetector wherein the carbon dots embedded in the film exhibit excitation fluorescence peak at a wavelength in the range of 420 nm<λ₂<500 nm, with the photodetector being selectively responsive to light illumination at a wavelength in said range;

A capacitive organic photodetector, wherein the carbon dots embedded in the film exhibit excitation fluorescence peak at a wavelength in the range of 500 nm<λ₃, with the photodetector being selectively responsive to light illumination at a wavelength in said range; and

A photodetector array comprising individual capacitive organic photodetectors as set out above.

The photodetector array enables a photodetection method to distinguish between different wavelengths of incident light, by illuminating the photodetector array with light and measuring variation in capacitance in the individual photodetectors, each of which is selective to wavelength subrange.

As pointed out above, the invention also relates to a capacitive photodetector which can distinguish circularly polarized light through enantioselective polymerization of the monomer of Formulas I/IA. Formation of a chiral polydiacetylene film on the surface of the electrode is achieved by irradiation with circular polarized visible light (abbreviated CPVL; generated for example, by 532 nm Nd:YAG laser) simultaneously with conventional UV irradiation, e.g., at 254 nm, with the intensity of the CPVL and UV light being in the ranges of 25 to 35 mWcm⁻² and 15 to 20 mWcm⁻², respectively. Synthesis of polydiacetylene films with irradiation by circular polarized UV light (CPUL) can also be applied to afford the opposite chiral PDA films.

Accordingly, an additional aspect of the invention includes a capacitive organic photodetector, comprising a polydiacetylene film-coated electrode with reducible sites along the polymer backbone and carbon dots embedded in the film, wherein the polydiacetylene shows chirality. The invention also provides a photodetector array comprising at least a first and second organic photodetectors, wherein the individual photodetectors comprise polydiacetylene films of opposite chirality. Such an array is used to carry out a photodetection method to determine circularly polarized light, comprising illuminating the photodetector array with the light and measuring variation in capacitance in the individual photodetectors, wherein strong and weak changes in the capacitances measured in the first and second photodetectors, respectively, indicate that the polarization of the incident light corresponds to the chirality of the polydiacetylene coating on the first photodetector, and wise versa. That is, a photodetector based on left-polymerized film shows strong and weak changes in capacitance when illuminated by left-handed CPVL and right-handed CPVL, respectively; and a photodetector based on right-polymerized film shows strong and weak changes in capacitance when illuminated by right-handed CPVL and left-handed CPVL, respectively.

A change in capacitance is expressed by ΔC/C (%) and can be determined as described in the experimental section below. The ratio between strong and weak photoinduced changes exhibited by PDA films with opposite chirality in response to CPVL illumination is at least >2, e.g., >3, for example, >4. For example, a strong change in capacitance may be >50%, whereas a weak change in capacitance is <15%, attesting to the ability of the photodetector array to respond selectively to illumination by circularly polarized light when light polarization matches the film chirality.

For example, the array comprises a pair of photodetectors with opposite film chirality, the first photodetector consisting of left hand-polymerized-Bis-ADA/C-dot film applied on an electrode surface and the second photodetector consisting of right hand-polymerized-Bis-ADA/C-dot film applied on an electrode surface. Illumination with different circularly polarized light (at a wavelength corresponding to the selected C-dots incorporated in the films as previously explained), results in different changes in capacitance at the two individual photodetectors, with a correlation between incident light polarization and sensor film chirality. That is, when the left-polymerized-Bis-ADA/C-dot photodetector is illuminated with left-handed CPVL, a significant capacitive response (>50%, e.g., 60%) is generated, while the right-handed-CPVL gives rise to lower capacitive signal (<20%, e.g., <10%) and vice versa, i.e., an opposite response to light polarization is observed in the case of right-polymerized-Bis-ADA/C-dot film.

The wavelength and intensity of the incident light beam illuminating the photodetector may be, for example, from 350 to 650 nm and >5 mW/cm² respectively (e.g., >10 mW/cm², e.g., >30 mW/cm², e.g., from 50 to 100 mW/cm²). Recordation of capacitance is done by connecting the coated electrode to a capacitance meter, such as an LCR meter.

### BRIEF DESCRIPTION OF THE DRAWINGS

FIG. 1 shows the main features of the invention, starting with the synthesis of bis(diacetylene) anthraquinone monomer, polymerization in the presence of C-dots on the surface of IDE, and the photoinduced capacitance increase in response to light illumination.

FIG. 2i shows the syntheses of different C-dots and their distinct fluorescence emissions. The normalized fluorescence excitation spectra are shown in FIG. 2ii.

FIGS. 3A and 3B show the ¹H NMR and ¹³C NMR spectra of the monomer Bis-ADA.

FIG. 4A shows the fabrication of the capacitive photodetector by drop-casting a solution of the Bis-ADA monomer and C-dots on the IDE, subsequent UV polymerization, SEM image of the obtained film, and thermochromism displayed by the film. FIGS. 4Bi, 4Bii and 4Biii show the SEM images of films based on comparative monomers (4Bi and 4Bii) and on the Bis(diacetylene) anthraquinone of the invention (4Biii).

FIG. 5 shows the capacitive photo-response of the polymerized-Bis-anthraquinone-diacetylene/carbon-dots photodetector. FIG. 5A: capacitance percentage change upon illumination of the polymerized-Bis-ADA/C-dot film-coated IDE with light pulses (generated by a solar simulator at 70 mW/cm²). Photoinduced capacitance increase is apparent only in the case of polymerized-Bis-ADA film. FIG. 5B: temporal analysis of the light-induced capacitive signal recorded by polymerized-Bis-ADA/C-dot photodetector upon application of a light pulse. FIG. 5C: Light intensity dependence of the photoinduced capacitive signals; the power values of the illuminating light beam (in mW/cm²) are indicated. FIG. 5D: stability of the photoinduced capacitive signals after repeated application light pulses (250 msec on; 2000 msec off, generated by a solar simulator).

FIG. 6 shows the wavelength selectivity of the polymerized-Bis anthraquinone-diacetylene/carbon dots. FIG. 6A: photoinduced capacitive response upon light illumination at different wavelengths (i. 400 nm; ii. 500 nm; iii. 600 nm) of polymerized-Bis-ADA/C-dot films comprising blue C-dot (blue), green C-dot (green) or red C-dots (red). FIG. 6B: capacitance signal intensities recorded in polymerized-Bis-ADA/C-dot films comprising blue C-dot (blue), green C-dot (green) or red C-dots (red), upon illumination at different wavelengths. FIG. 6C: Principal components analysis (PCA) showing capacitive response cluster differentiation according to wavelength illumination.

FIG. 7 shows that chiral polymerized-Bis-anthraquinone-diacetylene/carbon-dot photodetector distinguishes circularly polarized light. FIG. 7A is a schematic illustration of the enantio-selective polymerization of Bis-ADA afforded by circularly polarized 532 nm Nd:YAG laser, applied simultaneously with non-polarized UV light (16 W UV lamp, λ=254 nm). FIG. 7B shows circular dichroism (CD) spectra of red polymerized-Bis-ADA/C-dot film after irradiation with left-handed circularly polarized visible light (CPVL) together with nonpolarized UV light (blue spectrum); right-handed CPVL and nonpolarized UV light (red spectrum) and nonpolarized UV light alone (black spectrum). FIG. 7C shows the measured capacitance response signal under illumination by left-handed-CPVL at 550 nm (blue bars) and right-handed CPVL at 550 nm (red bars) for (i) left-polymerized Bis-ADA and (ii) right polymerized Bis-ADA, both containing red C-dots.

FIG. 8 shows a mechanistic analysis of photo-induced capacitive sensing in the polymerized-Bis-anthraquinone/carbon dot films. FIG. 8A shows the normalized fluorescence spectra of the silicon substrate (brown spectrum), deposited C-dots (red spectrum), polymerized-Bis-ADA film (green spectrum) and polymerized-Bis-ADA/C-dot film (black spectrum); excitation was at 254 nm. FIG. 8B is the surface photovoltage spectra (SPS) of films comprising C-dots only (red spectrum), polymerized-Bis-ADA (green spectrum), and polymerized-Bis-ADA/C-dot film (black spectrum). FIG. 8C is a scheme showing the proposed mechanism of light-induced reduction of the quinone moieties in the polymerized-Bis-ADA by photo-excited electrons transferred from proximate C-dots. FIG. 8D is a cyclic voltammetry (CV) curve recorded for polymerized-Bis-ADA/C-dot in 0.1M tetrabutylammonium-hexafluorophosphate (NBu₄PF₆)/acetonitrile (CAN) deposited on a glassy carbon electrode at a scan rate of 100 mV/s.

FIG. 9 shows capacitance percentage change upon illumination of the polymerized Bis-ADA/C-dot (red, of the invention), AQ-DA/C-dot (black, comparative—see Example 2A) and AQ-DA-AQ/C-dot (blue, comparative—see Example 2B) film-coated IDE with light pulses (generated by a solar simulator at 70 mW/cm²).

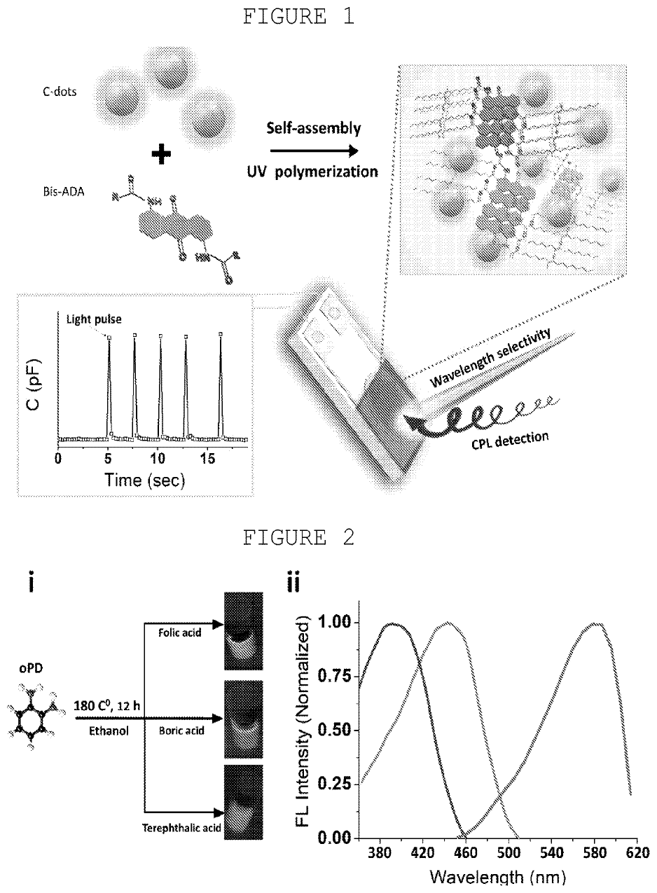

### EXAMPLES

### Materials

Oxalyl chloride (98%, Acros Organic), 1,5-Diaminoanthraquinone (90%, Alfa Aesar), o-phenylenediamine (oPD), 1-Aminoanthraquinone 97%, folic acid (FA), boric acid 99.5% (BA), terephthalic acid (TPA), Tetrabutylammonium hexafluorophosphate (NBu₄PF₆, 98%) and N,N-Dimethylformamide anhydrous 99.8% (Sigma Aldrich) were used as received. 10,12-tricosadiynoic acid (98%, Alfa Aesar) was purified prior to use by dissolving in chloroform and passing using a 0.8 μm syringe filter followed by solvent removal by rotatory evaporation. All organic solvents were purchased from Bio-Lab Ltd., Jerusalem, Israel. Interdigitated gold electrodes (Dimensions: 10×6×0.75 mm; glass substrate; Insulating layer: EPON SU8 resin; electrode material: Au; electrode thickness: 150 nm; microelectrode width: 10 μm, microelectrode gap: 10 μm; the number of fingers: 90 pairs) were purchased from MicruX Technologies (Oviedo, Spain).

### Preparations 1A-1C

### Synthesis of C-Dots

Synthesis of the C-dots was based on reported procedures for the construction of multi-colored C-dots through a scalable acid reagent engineering strategy (Wang, W. et al., Full-color fluorescent carbon quantum dots, supra).

### Blue C-Dots:

500 mg of o-phenylenediamine and 500 mg of folic acid were dissolved in a 10-ml ethanol solution. Subsequently, the solution was transferred into a 25-ml Teflon-lined stainless-steel autoclave and heated at 180° C. for 12 hours. After cooling to room temperature, the solution was filtered with a 0.22-μm microporous membrane, and the C-dots were further purified by dialysis in ethanol. C-dot powder was obtained for further characterization after evaporation.

### 1B: Green C-Dots

The procedure described above was repeated, but boric acid (500 mg) was reacted with the o-phenylenediamine.

### 1C: Red C-Dots

The procedure described above was repeated, but terephthalic acid (500 mg) was reacted with the o-phenylenediamine.

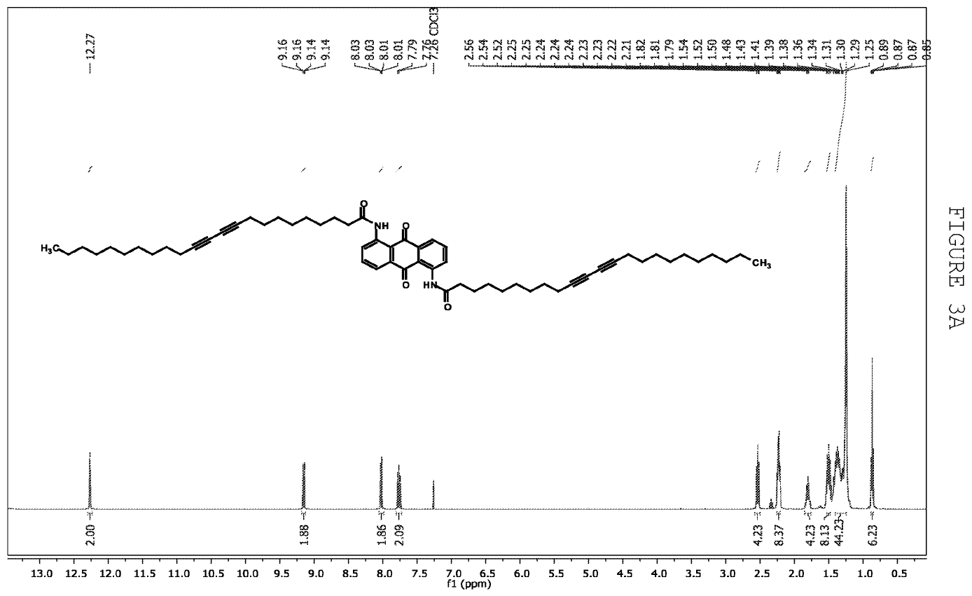

The synthesis of blue C-dots, green C-dots, and red C-dots through hydrothermal treatment of ortho-phenylenediamine in ethanol/acid mixtures as described above, and their normalized fluorescence spectra of the three C-dots produced are shown in FIG. 2. The syntheses solvothermal syntheses yielded C-dots exhibiting distinct colors (i.e., distinct fluorescence emissions; photographs showing the C-dot colors upon illumination at 365 nm are presented in FIG. 2i). The normalized fluorescence excitation spectra in FIG. 2ii underscore the specific wavelengths, in a broad spectral range, absorbed by the C-dots. Specifically, maximal fluorescence excitations at 400 nm, 440 nm, and 580 nm for blue C-dots, green C-dots, and red C-dots, respectively, were attained.

Fluorescence spectra of C-dot solutions were recorded using a fluorescence spectrophotometer (Horiba, Japan). Fluorescence emissions were measured at different excitation wavelengths ranging from 360 nm to 620 nm. The fluorescence was measured at a 90° angle relative to the excitation light. This geometry is used instead of placing the sensor at the line of the excitation light at a 180° angle to avoid interference of the transmitted excitation light.

### Example 1

### Preparation of Bis-ADA

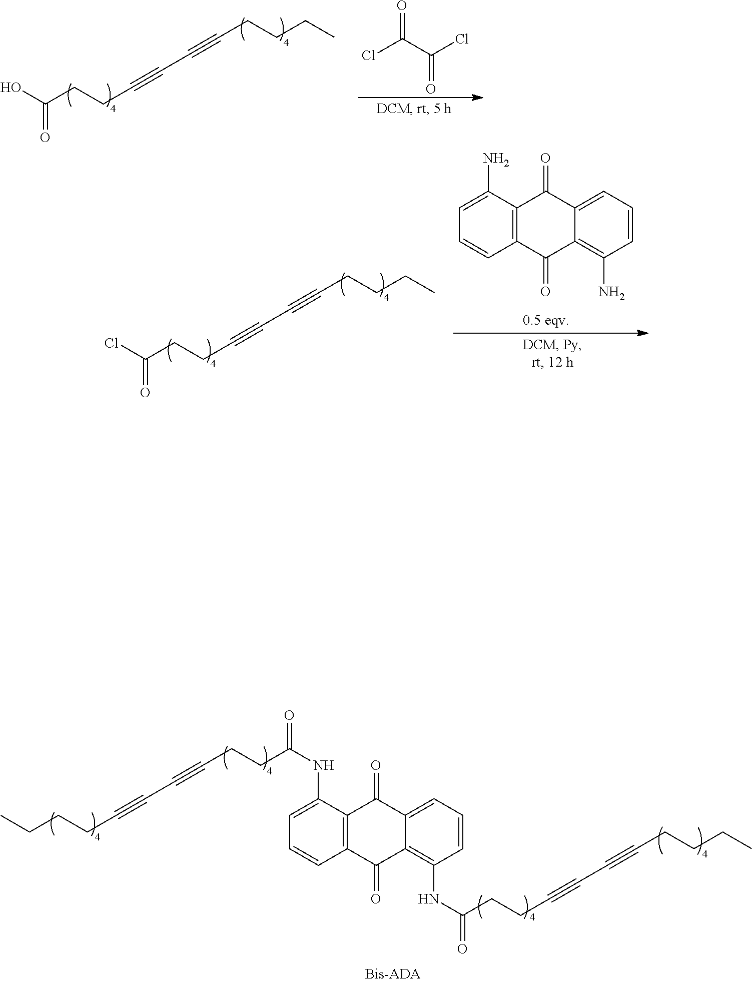

10,12-tricosadiynoic acid (1.04 g, 3.0 mmol) was dissolved in 20 mL dichloromethane (DCM) in a round-bottomed flask equipped with a magnetic stirrer. 1 mL of oxalyl chloride was added to the solution and stirred for 30 min at room temperature. N,N-dimethylformamide (DMF) was added to the solution in catalytic amounts (5-6 drops) and stirred for 4 hours. The solvents and excess oxalyl chloride were removed by a rotatory evaporator under a vacuum to give the corresponding acyl chloride. The residue was dissolved in 10 mL DCM and used directly in the next step. In a separate flask, 1,5-diaminoanthraquinone (0.357 g, 1.5 mmol) was dissolved in pyridine (20 mL) and dichloromethane (20 mL). Acyl chloride solution (from the previous step) was added dropwise to the reaction mixture and once the addition was completed the resultant solution was stirred at room temperature for another 12 hours. Upon completion of the reaction, the dichloromethane was removed from the reaction mixture under vacuum and 200 mL of doubly distilled water was added. The resultant precipitate was filtered and washed with 100 mL water twice. The residue was transferred into a flask containing 200 mL of ethanol, stirred in a hot bath for 10 minutes and allowed to cool followed by filtration. The precipitate was collected and dissolved in chloroform. The solvents were removed under vacuum, and the crude product was purified by column chromatography over silica gel using ethyl acetate/toluene (1:9) to give Bis-ADA as a yellow powder. (0.800 g, 60% yield).

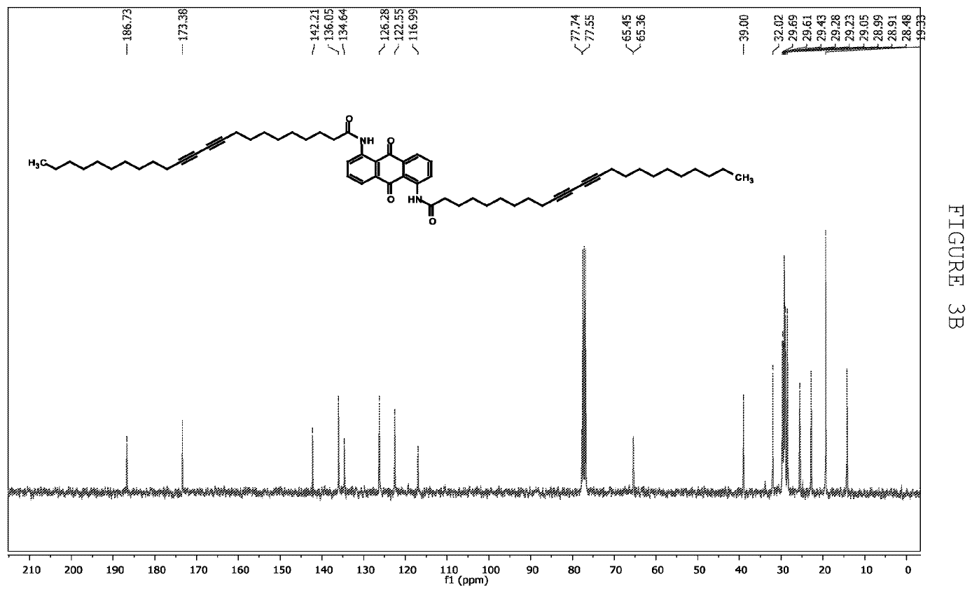

¹H and ¹³C Nuclear Magnetic Resonance (NMR) spectra were recorded. Flash chromatography was performed using silica gel (230-400 mesh) as the stationary phase. NMR spectra were recorded on Bruker DPX 400 instrument. The chemical shifts, given in ppm, are relative to the residual solvent peaks: ¹H NMR (400 MHz, CDCl₃) δ 12.27 (s, 2H), 9.15 (dd, J=8.6, 1.2 Hz, 2H), 8.02 (dd, J=7.7, 1.2 Hz, 2H), 7.85-7.65 (m, 2H), 2.54 (t, J=7.6 Hz, 4H), 2.25-2.21 (m, 8H), 1.85-1.75 (m, 4H), 1.53-1.48 (m, 8H), 1.40-1.24 (m, 44H), 0.87 (t, J=6.9 Hz, 6H). ¹³C NMR (100 MHz, CDCl₃) δ 186.7, 173.4, 142.2, 136.1, 134.6, 126.3, 122.6, 117.0, 77.7, 77.6, 65.5, 65.4, 39.0, 32.0, 29.7, 29.6, 29.4, 29.3, 29.2, 29.1, 29.0, 28.9, 28.5, 25.5, 22.8, 19.3, 14.2. MALDI-TOF m/z [M+Na]+ Calcd for C₆₀H₈₂N₂O₄+Na 917.617, found 917.632. ¹H NMR and ¹³C NMR spectrum are shown in FIGS. 3A and 3B, respectively.

### Examples 2A and 2B (Comparative)

### Preparation of Comparative Monomers: AQ-DA and AQ-DA-AQ

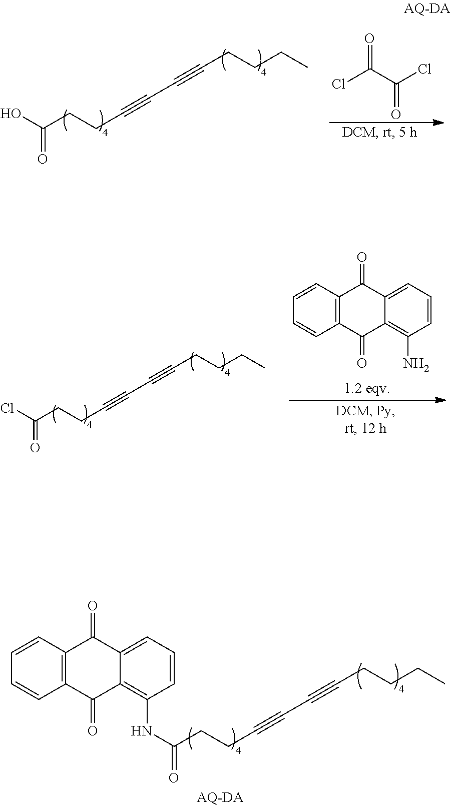

The procedure of Example 1 was repeated, starting with 10,12-Tricosadiynoic acid (1.04 g, 3.0 mmol) that is converted to the corresponding acyl chloride, followed by a reaction with 1-aminoanthraquinone (0.800 g, 3.6 mmol) at ~1:1 molar ratio, to give the corresponding product AQ-DA as a yellow powder (1.24 g, 75% yield).

¹H and ¹³C NMR spectra were recorded as described above. ¹H NMR (400 MHz, CDCl₃) δ 12.34 (s, 1H), 9.16 (dd, J=8.6, 1.2 Hz, 1H), 8.35-8.22 (m, 2H), 8.05 (dd, J=7.6, 1.2 Hz, 1H), 7.83-7.79 (m, 2H), 7.77 (dd, J=11.6, 4.6 Hz, 1H), 2.57-2.52 (m, 2H), 2.26-2.21 (m, 4H), 1.81 (dd, J=14.9, 7.4 Hz, 2H), 1.51 (dd, J=14.8, 7.2 Hz, 4H), 1.43-1.25 (m, 22H), 0.87 (t, J=6.9 Hz, 3H). ¹³C NMR (101 MHz, CDCl₃) δ 187.45, 182.85, 173.34, 142.40, 135.95, 134.48, 134.45, 134.20, 134.09, 132.98, 127.48, 127.18, 126.26, 122.51, 117.63, 77.75, 77.59, 65.45, 65.37, 39.05, 32.03, 29.70, 29.61, 29.44, 29.30, 29.23, 29.06, 28.99, 28.93, 28.49, 28.46, 25.58, 22.81, 19.34, 14.25. MALDI-TOF m/z [M+Na]⁺ Calcd for C₃₇H₄₅NO₃+Na 574.329, found 574.340.

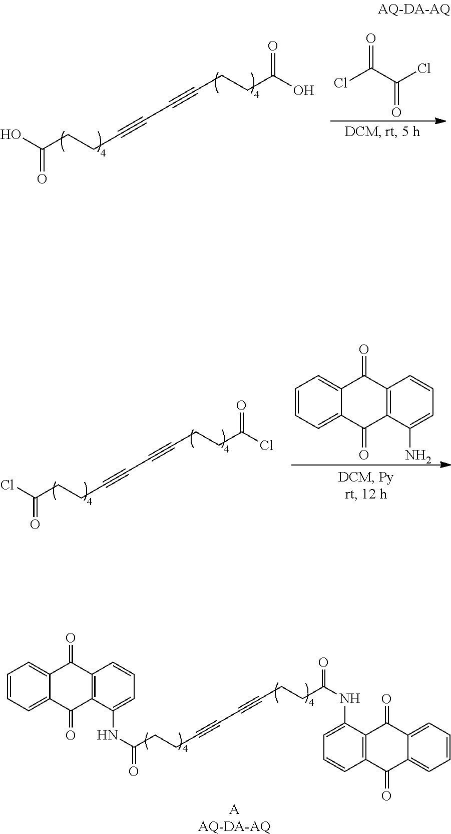

The procedure of Example 1 was repeated, starting with 10,12-Tricosadiynoic acid (1.04 g, 3.0 mmol) that is converted to the corresponding acyl chloride, followed by a reaction with 1-aminoanthraquinone (1.47 g, 6.6 mmol) at ~1:2 molar ratio, to give the corresponding product. Due to low solubility, the crude product is not subjected to column chromatography instead taken in hot acetone 200 mL, allowed to cool, and filtered to yield AQ-DA-AQ as an amorphous yellow solid (1.51 g, 65% yield).

¹H and ¹³C NMR spectra were recorded as described above. ¹H NMR (400 MHz, CDCl₃) δ 12.35 (s, 2H), 9.17 (dd, J=8.5, 1.2 Hz, 2H), 8.37-8.22 (m, 4H), 8.06 (dd, J=7.6, 1.2 Hz, 2H), 7.84-7.80 (m, 4H), 7.80-7.75 (m, 2H), 2.56 (t, J=7.6 Hz, 4H), 2.25 (t, J=7.0 Hz, 4H), 1.85-1.81 (m, 4H), 1.56-1.52 (m, 4H), 1.45-1.34 (m, 18H). ¹³C NMR (101 MHz, CDCl₃) δ 187.44, 182.84, 173.34, 142.40, 135.95, 134.48, 134.45, 134.19, 134.09, 132.97, 127.48, 127.18, 126.25, 122.51, 117.63, 77.65, 65.45, 39.04, 29.30, 29.06, 28.93, 28.45, 25.58, 19.34. MALDI-TOF m/z [M+Na]⁺ Calcd for C₅₀H₄₈N₂O₆+Na 795.340, found 795.366.

### Example 3

Fabrication and Characterization of a Capacitive Photodetector Consisting of an Interdigitated Electrode Coated with Polydiacetylene-Anthraquinone/Carbon Dots

7.5 mg of C-dot were mixed with 15 mg of Bis-ADA monomer dissolved in 1 ml of toluene. The solution was then drop-casted (10 μL) on the surface of the IDE and dried for 2 h at room temperature. The resultant coated electrodes were further irradiated for 2 min with a UV lamp (254 nm, 16 W) to induce PDA cross-linking in the ambient atmosphere. The color of the deposited composite film changed from yellowish-green color to dark brown, further transforming to red after annealing on a hot plate (80° C. for 1 min) (five electrodes were employed in each experiment reported herein).

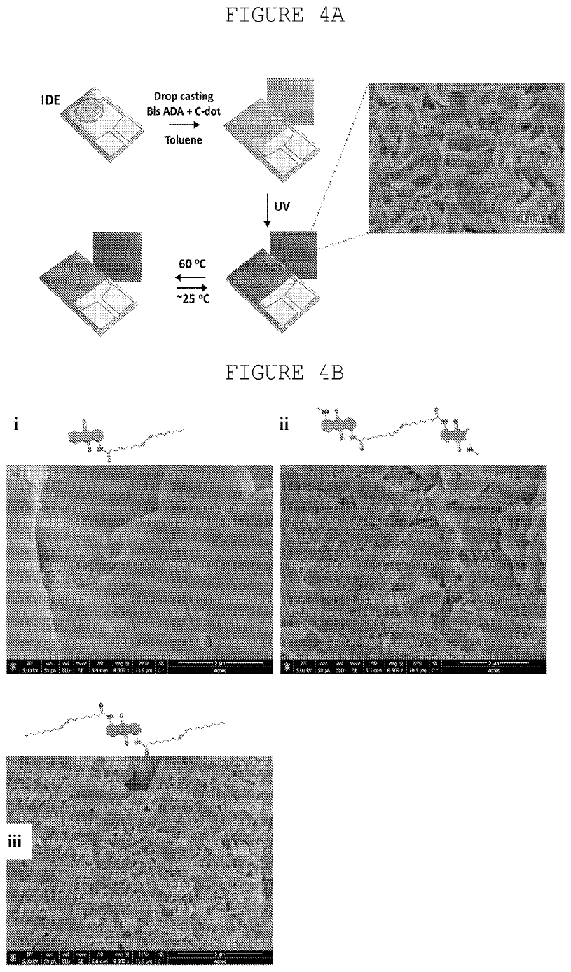

Fabrication of the capacitive photodetector by drop-casting the Bis-ADA monomer and C-dots on the interdigitated electrode and subsequent UV polymerization as described above, is shown in FIG. 4A. A representative scanning electron microscopy (SEM) image of the polymerized-Bis-ADA/C-dot film is also presented (inset), revealing a sheet-like morphology of the aligned polydiacetylene network (SEM images of the films on the IDE were acquired after air-drying overnight. The dried sample was covered with 5 nm of Au sputtering. The samples were imaged using a FEI Verios, SEM (Thermo Fisher Scientific, XHR 460L). The images were viewed at different magnifications, in an acceleration voltage of 5 kV).

The polymerized-Bis-ADA/C-dot film exhibits reversible thermochromism (FIG. 4A, bottom). Specifically, upon increasing the temperature to 60° C., the film transformed into a reddish color (FIG. 4A, bottom left), reflecting the blue-red phase transition of the PDA network. The polymerized-Bis-ADA/C-dot film reverted to its original green-brownish color at 25° C. (FIG. 4A, bottom).

Comparative electrodes were prepared by drop-casting a monomer and C-dots on interdigitated electrodes and subsequent UV polymerization as described above. That is, to polymerize the monomer of Example 2A, i.e., diacetylene-anthraquinone consisting of single substituted sidechains or the monomer of Example 2B, i.e., disubstituted diacetylene with anthraquinones on both sides. The polymerization products of the comparative monomers produce films exhibiting significantly different morphology as compared to the sheet-like structure of the polymerized monomer of the invention, i.e., Bis-ADA, as clearly shown by the SEM images in FIG. 4B (4B(i): polymerized AQ-DA/C-dot on IDE surface, 4B(ii): polymerized AQ-DA-AQ/C-dot on IDE surface and 4B(iii) polymerized Bis-ADA/C-dot on IDE surface (SEM images acquired as previously described)).

In the next set of Examples, UV-Vis absorbance spectra, fluorescence spectra and capacitive measurements are reported.

UV-vis absorbance spectra were recorded on a Thermo Scientific Evolution 220 spectrophotometer in the range of 300-800 nm at room temperature.

Fluorescence spectra of the deposited films on a silicon wafer were acquired on a Newport Corp. MS257 spectrometer equipped with a Si CCD detector and long-pass order-sorting filters. A 1-mW He—Cd laser was used for excitation.

Capacitive photo-response measurements of the polymerized C-dot-Bis-ADA-IDE devices were carried out using an LCR meter (Keysight Technologies, E4980AL Precision LCR Meter). For the light source, a white light emitting diode (LED, Chanzon, China) operating in a wavelength range of 380-800 nm and mounted LED sources with wavelength centered at 300 nm (UV), 365 nm (UV), 405 nm (violet), 450 nm (blue), 505 nm (cyan), 554 nm (green), 600 nm (orange), and 645 nm (red) LEDs from Thorlabs were used. Light intensity was measured using a power meter (PM100, Thorlabs) while applying the illumination. Capacitive measurements were performed at room temperature upon exposure of devices consisting of polymerized Bis-ADA/C-dot on IDE surface to the different light sources. Capacitance values were recorded after reaching a clear baseline, collecting the data every 0.03 sec. Thorlabs DC2200a LED driver was used in the pulsed light experiments. The IDE photoinduced capacitance depends on variations of the dielectric constant of the deposited film. The capacitance of the interdigital electrode was defined by the Equation:

$$C = η {ε}_{o} {ε}_{r} \frac{l t}{d} \qquad ( 1 )$$

where C is the capacitance in farads (F), η is the number of fingers (90 in the IDE employed here), ε₀ is the permittivity of free space (ε₀=8.854×10⁻¹² F/m), εᵣ is the relative permittivity, also known as the dielectric constant, t is the thickness of interdigital electrodes, l is the length of interdigital electrodes and d is the distance between the electrodes. The capacitance response of the sensors—ΔC—was defined as C_{light}−C_{dark}, where C_{light} and C_{dark} are the capacitance value after illumination and the capacitance baseline value measured under dark conditions, respectively. Capacitance percentage change was calculated to compare between the electrodes.

### Example 4

Capacitive Photodetection Properties of an Interdigitated Electrode Coated with Polydiacetylene-Anthraquinone/Carbon Dots

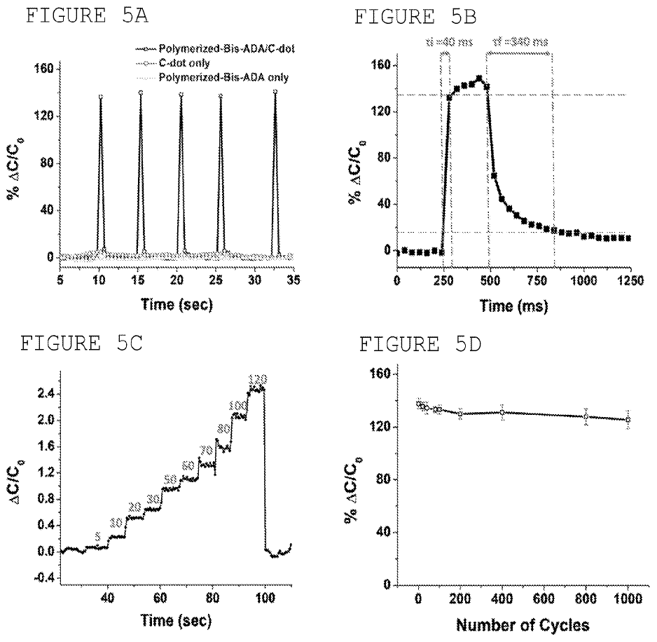

FIG. 5 depicts the capacitance changes induced upon illuminating the polymerized-Bis-ADA/C-dot photodetector, using light generated by a solar simulator (wavelength range of 300-2000 nm; illumination intensity 70 mW/cm²). FIG. 5A shows the capacitance recorded (presented as percentage changes) upon exposure to light pulses (500 μsec square waves, 70 mW/cm² peak-to-peak intensities). Capacitive transformations that traced the light pulses were recorded in the case of IDE coated with polymerized-Bis-ADA/C-dot films. In contrast, negligible capacitance changes were observed upon application of light pulses using control electrodes that were coated with just C-dots, or only with polymerized-Bis-ADA (FIG. 5A). Accordingly, the data in FIG. 5A confirm that the capacitive photo response occurred only in the presence of both C-dots and Bis-ADA polymer.

FIG. 5B highlights the sensitivity and temporal resolution of the photo-induced capacitance signals. The polymerized-Bis-ADA/C-dot photodetector was exposed to a light pulse having 250 ms duration (70 mW/cm², generated by a solar simulator), and the capacitive response was recorded. The % ΔC/C₀ versus time plot indicates ~40 msec rise time and ~350 msec fall time with a large capacitance change of ~150%, demonstrating pronounced sensitivity.

The sensitivity and stability of the photo-induced capacitive signals are further underlined in FIG. 5C. The graph in FIG. 5C tracks the relative capacitance change (ΔC/C₀) recorded upon continuously illuminating the polymerized-Bis-ADA/C-dot photodetector using different light intensities (illumination intensities in mW/cm² using a white-emitting LED are indicated). FIG. 5C reveals the stability of the photo response at varied light intensities, reflected in the relative plateaus in the time-dependent capacitive signals. In addition, an experimentally significant response was apparent even upon illuminating the sensor with low, 5 mW/cm² light beam power. The pulse cycle analysis in FIG. 5D further underscores the excellent stability and reproducibility of the polymerized-Bis-ADA/C-dot photodetector. Indeed, less than 5% capacitive signal variation is apparent even after the application of more than 1000 on/off light pulses (250 msec light pulse duration).

### Example 5

### Photo-Response of Polydiacetylene-Anthraquinone/C-Dots Films Comprising C-Dots Exhibiting Absorbance at Different Wavelengths

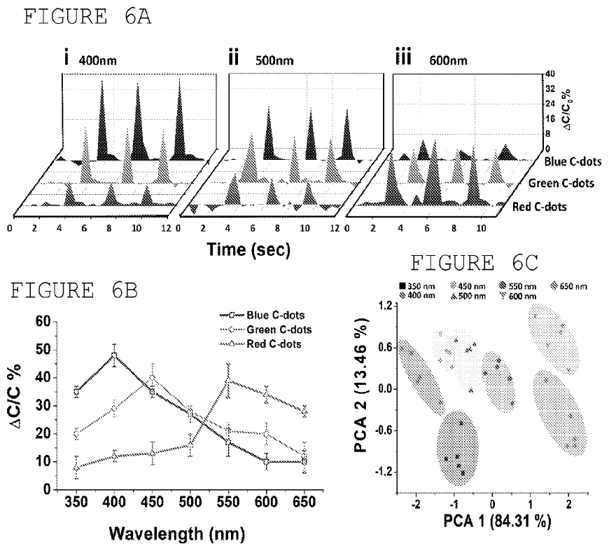

Three polymerized-Bis-ADA/C-dot photodetectors, each comprising C-dots exhibiting a different light absorbance/emission profile (i.e., blue C-dots, green C-dots, or red C-dots) were prepared and illuminated at different light wavelengths (generated by single-color cold-mounted LEDs). The capacitance changes recorded are shown in FIG. 6A.

The results indicate that the capacitance changes depend on the wavelengths of incident light. That is, the wavelength dependence is directly linked to the absorbance wavelengths of the C-dots embedded in the polymerized-Bis-ADA matrix. For example, illumination with a 400 nm light gave rise to a maximal capacitance response—of around 50%—in the case of the blue polymerized-Bis-ADA/C-dot photodetector (FIG. 6Ai, blue signals). This electrode, in comparison, produced lower capacitive signals (~25%) when illuminated by the LED exhibiting a wavelength of 500 nm (FIG. 6Aii), and even lower still (<10%) in the case of 600 nm light pulses (FIG. 6Aiii). Similarly, the most pronounced capacitance change upon illumination at 500 nm was recorded for the green-polymerized-Bis-ADA/C-dot photodetector (FIG. 6Aii), while the polymerized-Bis-ADA/red C-dot photodetector produced the highest capacitive signal upon illumination at 600 nm (FIG. 6Aiii). The % ΔC/C₀ versus wavelength plot in FIG. 6B illustrates the overall wavelength dependence of the capacitive response, underlining the key role of the C-dot absorbance wavelength in determining the sensor response.

The capacitive response data obtained upon illumination of the three electrodes (each coated with a polymerized-Bis-ADA/C-dot film comprising a different C-dot species) with various monochromatic pulses were classified according to principal component analysis (PCA) (FIG. 6C). The PCA graph in FIG. 6C underscores the extraordinary capacity of the polymerized-Bis-ADA/C-dot photodetector to distinguish among wavelengths of illuminating light. Specifically, FIG. 6C depicts the score plot in the first two principal component spaces in which PCl accounts for the greatest total variation (84.31%), and each point represents three independent capacitive measurements. Importantly, the clustering of the experimental datapoints in the PCA plot shows almost no overlaps between the wavelengths tested. Indeed, the photo-induced capacitive “fingerprints” depicted in FIG. 6C, obtained with just three electrodes, underscore the power of the photodetector to distinguish wavelengths of incident light. Increasing the number of the photodetector array elements (i.e., preparing electrodes deposited with C-dots having different light absorbance profiles) would enhance the wavelength discrimination capabilities.

### Example 6

### Chiral Polydiacetylene-Anthraquinone/Carbon Dots Photodetector Distinguishes Circularly Polarized Light

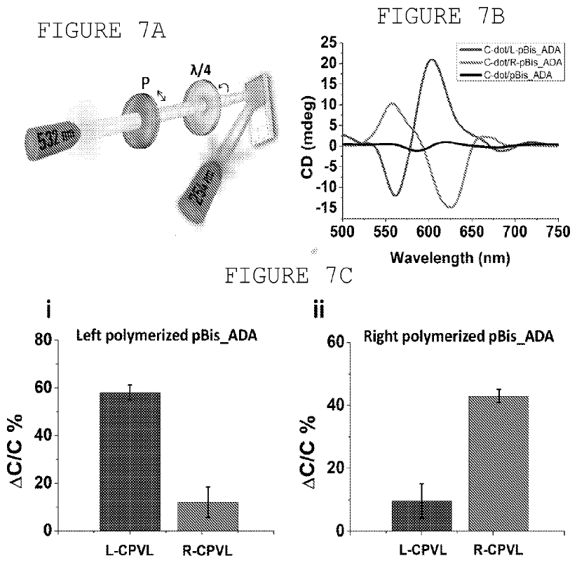

Two polymerized-Bis-ADA/C-dot photodetectors were prepared using the casting procedure described above. An enantio-selective polymerization reaction was then carried out through irradiation with circularly polarized visible light (CPVL) generated by 532 nm Nd:YAG laser, applied simultaneously with non-polarized UV light (16 W UV lamp, λ=254 nm), using the apparatus shown schematically in FIG. 7A (see also G. Yang, L. Han, H. Jiang, G. Zou, Q. Zhang, D. Zhang, P. Wang, H. Ming, Chemical Communications 2014, 50, 2338). Briefly, CPVL was created by passing a laser light (532 nm Nd:YAG Semiconductor laser, light intensity 30 mWcm²) through a linear polarizer plus a λ/4 waveplate. Chiral Polymerization was applied through simultaneous irradiation with CPVL and non-polarized UV light (16 W UV lamp, λ=254 nm), to form the enantio-selective polymerization.

Circular dichroism (CD) spectra were recorded in the range of 500-750 nm at room temperature on a Jasco J-715 spectropolarimeter, using an optical path length of 0.1-mm via quartz plate cuvettes. The plate cuvettes are coated with a thin film composed of C-dot/L-polymerized-Bis-ADA, C-dot/R-polymerized-Bis-ADA, and a control of C-dot/polymerized-Bis-ADA. The CD spectra were measured by placing the film perpendicular to the light path using JASCO CD spectrometer J-810.

The spectra are shown in FIG. 7B. It is seen that the interaction between the CPVL and the Bis-ADA film effectively imposed chiral order in the chain-propagating process. FIG. 7B confirms that the two polymerized-Bis-ADA/C-dot films exhibited opposite chirality. Essentially, the circular dichroism (CD) spectra in FIG. 7B display positive and negative Cotton effects, respectively, appearing at 560 nm and 615 nm with a crossover at 580 nm (blue and red spectra, respectively). The distinctive CD signals are ascribed to chiral polydiacetylene. Importantly, not inducing chirality of polymerized-Bis-ADA/C-dot films (using non-polarized light at 254 nm for polymerization) did not give rise to CD signals (FIG. 7B, black spectrum).

The sensitivity of the chiral polymerized-Bis-ADA/C-dot photodetector to light polarization is demonstrated in FIG. 7C. The bar diagram in FIG. 7C shows the capacitive responses of left-polymerized-Bis-ADA/C-dot photodetector and right-polymerized-Bis-ADA/C-dot photodetector, respectively, upon illumination with different circularly polarized light (illumination wavelength was 550 nm; C-dots incorporated in the films were red C-dots exhibiting maximal absorbance at 580 nm). Indeed, the capacitance response recorded in the experiments reveals a matching between light polarization and sensor film chirality. For example, when the left-polymerized-Bis-ADA/C-dot photodetector was illuminated with left-handed CPVL, a pronounced capacitive response of 60% was recorded, while the right-handed-CPVL gave rise to a lower capacitive signal (~10%, FIG. 7Ci). The opposite response to light polarization was observed in the case of the right-polymerized-Bis-ADA/C-dot film (FIG. 7Cii).

### Example 7

### Mechanistic Analysis

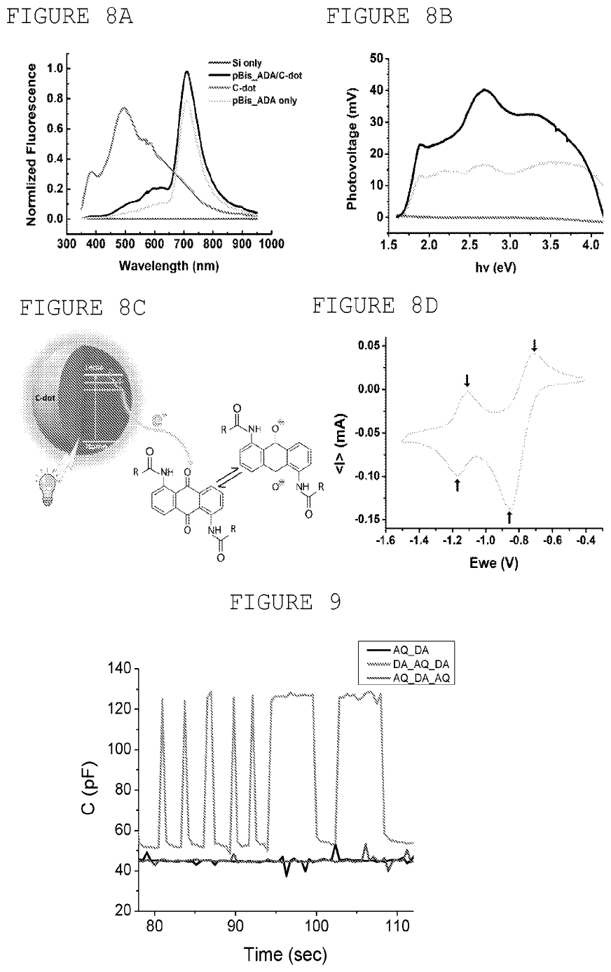

FIG. 8 presents spectroscopic and electrochemical experiments designed to decipher the mechanistic basis of the capacitive photo-response of the polymerized-Bis-ADA/C-dot system. The normalized fluorescence (NF) spectra in FIG. 8A (excitation at 254 nm) reflect different radiative transitions of the photo-induced electrons in the films. Notably, the NF peak at around 715 nm, recorded for the polymerized-Bis-ADA/C-dot film (FIG. 8A, black spectrum), exhibits higher intensity compared to a polymerized-Bis-ADA film alone (FIG. 8A, green spectrum), indicating that C-dot immobilization gave rise to an increase in the fluorescence intensity of the composite film. In parallel, the major NF signal of the C-dot-only film at around 500 nm (FIG. 8A, red spectrum) almost completely disappeared in the mixed polymerized-Bis-ADA/C-dot film, likely accounting for non-radiative electron transfer to the polymer. Overall, the NF data in FIG. 8A indicates that electrons generated in the C-dots through photon absorption are transferred to the surrounding polymerized-Bis-ADA matrix through non-radiative processes.

Surface photovoltage spectroscopy (SPS) analysis of different photodetector film configurations is depicted in FIG. 8B. SPS tracks changes in the surface voltage as a function of incident photon energy; the technique provides information on charged carrier transitions upon illumination. SPS was acquired on films deposited on Si templates having a ground contact on their periphery. The photovoltage signal was acquired using a Besocke Delta Phi Gmbh Kelvin probe from samples placed in a dark Faraday cage at room temperature. The samples were illuminated using a 300 W Xe light source monochromatized by Newport Corp. MS257 monochromator. To prevent illumination by second-order diffractions of the grating, the monochromatized light was further filtered by order-sorting long-pass filters. The photon flux was kept constant throughout the entire spectral acquisition by means of a variable slit operated in a closed control loop.

The results are shown in FIG. 8B, indicating differences between the polymerized-Bis-ADA/C-dot film and control samples (C-dots alone, polymerized-Bis-ADA alone). The observed photovoltage recorded in the case of the polymerized-Bis-ADA/C-dot and polymerized-Bis-ADA films indicates photoexcitation and spatial displacement of charged carriers upon illumination. The barely discerned photovoltage in the case of the C-dot-only film attests to the key role of the polymerized-Bis-ADA framework in carrier generation. Pronounced (positive) photovoltage was recorded for the polymerized-Bis-ADA/C-dot film, likely accounting for the photo-excited electrons transferred from the C-dots to the polymer matrix (e.g., FIG. 8A) and increasing the abundance of charge carriers.

FIG. 8C portrays the proposed mechanism accounting for the photo-induced capacitance changes in the polymerized-Bis-ADA/C-dot system, based upon the photo-induced capacitive response data shown above and spectroscopic experiments in FIGS. 8A-B. Photons absorbed by the C-dots excite electrons between the HOMO and LUMO levels. The excited electrons consequently migrate to the proximate anthraquinone-polydiacetylene matrix. The anthraquinone units constitute efficient electron acceptors, undergoing redox transformations.

Cyclic voltammetry analysis of polymerized-Bis-ADA/C-dot in an aprotic solvent mixture confirms the occurrence of reduction processes generating the Bis-ADA dianion. Cyclic voltammetry (CV) measurements were performed in a three-electrode configuration, with a polymerized sample on a glassy carbon electrode (GCE) as the working electrode, Pt wire as the counter electrode, and Ag/AgCl as the reference electrode. 0.1M NBu₄PF₆ in acetonitrile was used as the aprotic electrolyte, and the measurements were performed at a scan rate of 100 mV/s at a voltage range of −0.4 V to −1.5 V vs. Ag/AgCl. The experiments were performed on a BioLogic SP-150 instrument (Seyssinet-Pariset, France). Prior to material deposition, the glassy carbon electrode was cleaned by polishing with 0.25 μm diamond polishing compound, then rinsing with copious amounts of DI water, followed by a second polish with a 0.05 μm alumina slurry and a final rinse with DI water.

The CV curve in FIG. 8D demonstrates that the anthraquinone is electrochemically reduced in a two-step process (indicated by the arrows), usually observed in the CV of quinones. The electric potential values recorded for the reduction peaks are at −0.86 V and −1.18 V and the oxidation peaks are at −1.10 V and −0.70 V, indicating the formation of the single anion and subsequent dianion. FIG. 8D thus highlights a key outcome of electron excitation and consequent electron transfer from the C-dots to the Bis-ADA, specifically the reduction of the anthraquinone residues to anthraquinone-dianions.

The Bis-ADA→Bis-ADA²⁻ reduction process due to photoinduced electron transfer from the C-dots likely corresponds to the significant, rapid capacitance changes recorded in the polymerized-Bis-ADA/C-dot system. Since the dielectric constant (and concomitant capacitance measured) depends upon the polarizability of the molecules in the medium, the formation of the doubly charged anthraquinone appears to result in greater dielectric constants and higher capacitance, as depicted in the photo-induced capacitive results reported above. The unique organization of the Bis-ADA constituents, particularly the alignment of the anthraquinone headgroups, aids electron transfer from the C-dots and the consequent reduction of the quinones. In contrast, the comparative electrodes described in Example 3, i.e., polymerized AQ-DA/C-dot on IDE surface and polymerized AQ-DA-AQ/C-dot on IDE surface, do not show photoinduced capacitance transformations—see FIG. 9, showing photoinduced capacitance increase only in the case of polymerized-Bis-ADA film. The comparative electrodes lack the sheet-like structure of polymerized Bis-ADA, which accounts for the aligned anthraquinone organization and concomitant efficient electron transport.

The mechanistic analysis in FIG. 8 also accounts for the sensitivity of the polarized polymerized-Bis-ADA/C-dot to circularly polarized light (Example 6 and FIG. 7). In the case of the polymerized-Bis-ADA/C-dot photodetector, the polydiacetylene chirality introduced in the polymerization process produces a CPVL filtering capacity.

Essentially, when the circular polarization of the incident light matches the chirality of polymerized-Bis-ADA, the effective absorbance of light energy accounts for more efficient excitation of the embedded C-dots, giving rise to anthraquinone reduction and more pronounced change of the recorded capacitance, apparent in FIG. 7C.

1)-34) (canceled)

35) A bis(diacetylene) anthraquinone compound of Formula IA: 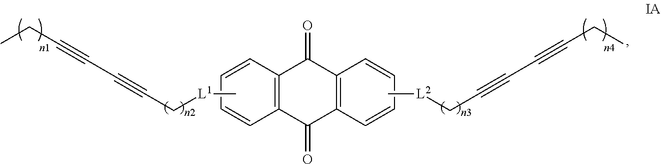
wherein n₁, n₂, n₃ and n₄ are integers selected independently from 3 to 12 and each of L¹ and L² is independently a linkage.

36) The bis(diacetylene) anthraquinone compound according to claim 35, wherein each of L¹ and L² is an amide linkage.

37) Symmetric bis(diacetylene) anthraquinone compound according to claim 36, wherein n₁=n₄ and n₂=n₃, having the structure:

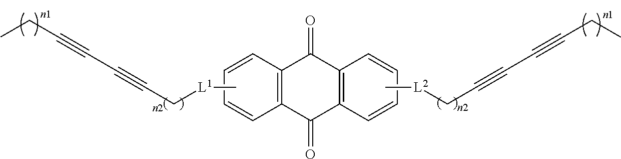

38) The bis(diacetylene) anthraquinone compound according to claim 37:

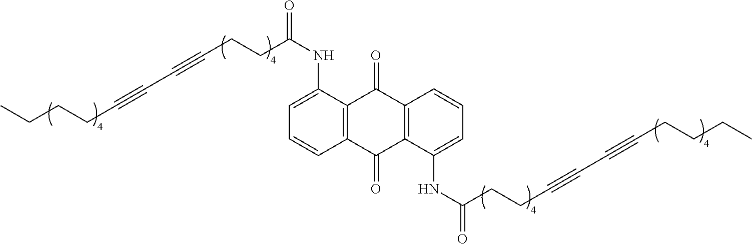

39) A process of preparing bis(diacetylene) anthraquinone compound of Formula IA comprising a reaction between diacetylene monomer of Formula 2 and anthraquinone of Formula 3: 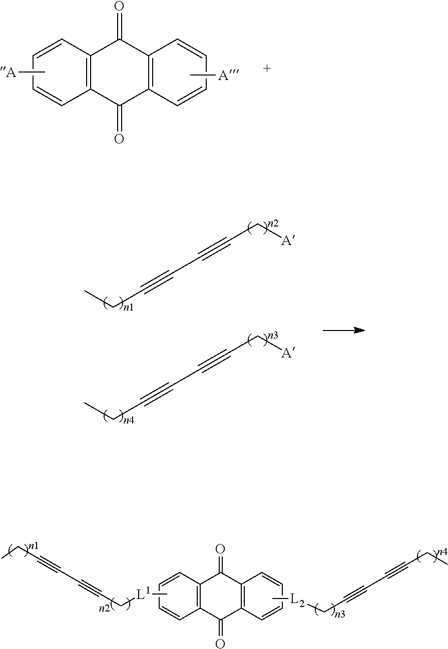
wherein A′, A″ and A″′ denote functional groups such that A′ participate in linkage formation reactions with A″ and A″′ to create the linkages denoted L¹ and L², which may be the same or different and n₁, n₂, n₃ and n₄ as defined in claim 35.

40) The process of preparing bis(diacetylene) anthraquinone compound of Formula IA according to claim 39: 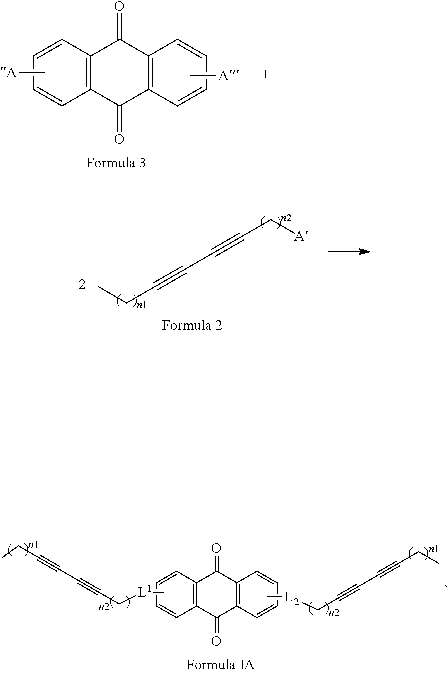
wherein A″ is —NH₂, A″′ is —NH₂, A′ is —C(O)Cl and each of L¹ and L² is amide bond —NH—C(O)—.

41) Polydiacetylene, comprising a repeat unit corresponding to the bis(diacetylene) anthraquinone monomer of Formula IA as defined in claim 35.

42) A process for preparing a capacitive organic photodetector, comprising the step of polymerizing on a surface of an electrode a reducible diacetylene monomer in the presence of carbon dots, thereby forming a polydiacetylene film coating onto the electrode, with the carbon dots being embedded in the film.

43) The process according to claim 42, wherein the reducible diacetylene monomer is bis(diacetylene) compound of the general formula:
CH₃—(CH₂)ₙ₁—C≡C—C≡C—(CH₂)ₙ₂-L¹-X-L²-(CH₂)ₙ₃—C≡C—C≡C—(CH₂)ₙ₄—CH₃, wherein X is a reducible aromatic group, each of L¹ and L² is an independently selected linkage and n₁, n₂, n₃ and n₄ are independently selected integers from 3 to 12.

44) The process according to claim 43, wherein X is a reducible polycyclic aromatic group consisting of two or more aromatic rings fused together, with at least one ring bearing one or more oxo (═O) groups attached to ring carbon(s), and each of L¹ and L² is an amide bond.

45) The process according to claim 44, wherein the bis(diacetylene) compound undergoing polymerization is represented by Formula IA:

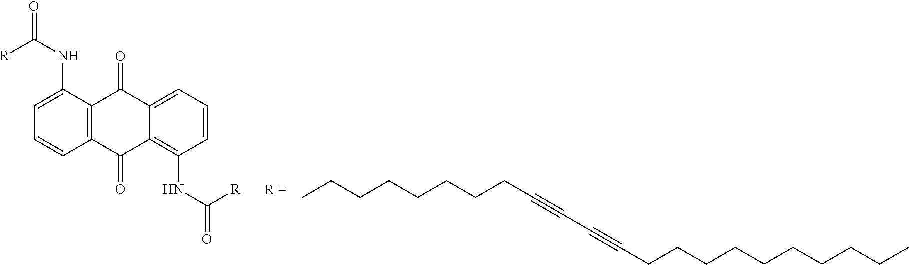

46) The process according to claim 42, for preparing a capacitive organic photodetector showing a wavelength-dependent increase in capacitance in response to light illumination, comprising incorporating into the film carbon dots that exhibit excitation fluorescence peak at a wavelength in the range of 300 nm<λ₁<420 nm, 420 nm<λ₂<500 nm or 500 nm<λ₃.

47) The process according to claim 42, comprising combining the diacetylene monomer and the carbon dots in an organic solvent to form a polymerizable mixture, applying the mixture by casting onto the electrode surface, followed by polymerization by irradiation with non-polarized UV light to create the polydiacetylene film, and optionally, simultaneously with the irradiation by non-polarized UV light, an irradiation with polarized light is carried out to create chiral polydiacetylene film.

48) A capacitive organic photodetector comprising a polydiacetylene film-coated electrode with reducible sites along the polymer backbone and carbon dots embedded in the film.

49) The capacitive organic photodetector of claim 48, wherein the backbone of polydiacetylene contains a reducible polycyclic aromatic group in the repeat unit.

50) The capacitive organic photodetector of claim 49, wherein the polycyclic aromatic group is a quinone derivative.

51) The capacitive organic photodetector of claim 50, wherein the repeat unit comprises: 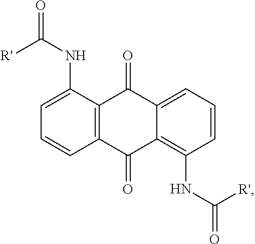
wherein R′ indicates a straight hydrocarbon chain corresponding to the polydiacetylene.

52) The capacitive organic photodetector of claim 48, showing wavelength-dependent capacitance change in response to light illumination.

53) The capacitive organic photodetector of claim 52, wherein the carbon dots embedded in the film exhibit excitation fluorescence peak at a wavelength in the range of 300 nm<λ₁<420 nm, with the photodetector being selectively responsive to light illumination at the wavelength in said range; or
the carbon dots embedded in the film exhibit excitation fluorescence peak at a wavelength in the range of 420 nm<λ₂<500 nm, with the photodetector being selectively responsive to light illumination at the wavelength in said range; or
the carbon dots embedded in the film exhibit excitation fluorescence peak at a wavelength in the range of 500 nm<λ₃, with the photodetector being selectively responsive to light illumination at the wavelength in said range.

54) A photodetector array comprising individual capacitive organic photodetectors according to claim 53.

55) A photodetection method to distinguish between different wavelengths of incident light, comprising illuminating the photodetector array of claim 54 with the light and measuring variation in capacitance in the individual photodetectors, each of which is selective to wavelength subrange.

56) The capacitive organic photodetector of claim 48, wherein the polydiacetylene shows chirality.

57) A photodetector array comprising at least a first and second organic photodetectors as defined in claim 56, wherein the individual photodetectors comprise polydiacetylene films of opposite chirality.

58) A photodetection method to determine circularly polarized light, comprising illuminating the photodetector array of claim 57 with the light and measuring variation in capacitance in the individual photodetectors, wherein strong and weak changes in the capacitances measured in the first and second photodetectors, respectively, indicate that the polarization of the incident light corresponds to the chirality of the polydiacetylene coating on the first photodetector, and vice versa.
